(19) United States
(12) Patent Application Publication
(10) Pub. No.: US 2018/0223075 A1
(43) Pub. Date: Aug. 9, 2018

(54) PIPE FOR THE TRANSPORT OF WATER HAVING IMPROVED RESISTANCE TO CHLORINATED DISINFECTANTS

(71) Applicant: SABIC GLOBAL TECHNOLOGIES B.V., BERGEN OP ZOOM (NL)

(72) Inventors: Mark Johannes Boerakker, Eindhoven (NL); Sarah van Mierloo, Opglabbeek (BE); Patrick Elisabeth Luc Voets, Born (NL)

(21) Appl. No.: 15/750,077

(22) PCT Filed: Jul. 14, 2016
(86) PCT No.: PCT/EP2016/066795

§ 371 Date: Feb. 2, 2018

(30) Foreign Application Priority Data
Aug. 4, 2015 (EP) 15179628.1

(51) Int. Cl. C08K 5/134; C08K 5/526; C07C 69/54; F16L 9/12; F17D 3/12

(52) U.S. Cl. CPC C08K 5/134; C08K 5/526; F17D 3/12; F16L 9/12; C07C 69/54

(57) ABSTRACT

The invention relates a pipe for drinking water distribution with improved resistance to chlorinated disinfectants characterised in that the pipe is produced with a polymer composition comprising a polyolefin and a bisphenol monoester.

The present invention relates to a pipe for the transport of water produced with a polymer composition comprising a polyolefin such as polyethylene and polypropylene. The pipe has improved resistance to chlorinated disinfectants.

Pipes for the transport of gas, for sanitation and for water supply may be produced with for example bimodal polyethylene compositions. Pipes have a very good resistance to water however their lifetime is shortened when the pipes come into contact with disinfectants which are often added to water for hygienic reasons. The chlorine dioxide used as disinfectant in water degrades most materials including polyethylene (Colin, Aging of polyethylene pipes transporting drinking water disinfected by chlorine dioxide, part I, Chemical aspects; Polymer engineering and Science 49(7); 1429-1437; July 2009). Other chlorinated solvents are for example chloramine and chlorine. It is known in the art to apply additives for example antioxidants and stabilizers to prevent said degradation. Several types of additives are proposed to protect polymers during processing and to achieve the desired end-use properties. However, appropriate combinations of stabilizers have to be carefully selected, depending on the desired final properties the polymeric article should have.

It is the object of the present invention to provide a pipe with improved service lifetime for the transportation of water containing chlorinated disinfectants, for example chlorine dioxide, chloramine and chlorine.

The pipe for the transport of water with resistance to chlorinated disinfectants according to the invention is produced with a polymer composition comprising polyethylene or propylene and a bisphenol monoester represented by Formula 1:

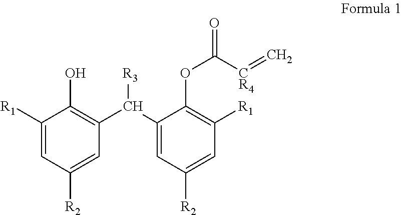

wherein each R₁ and R₂ independently represents an alkyl group having 1 to 5 carbon atoms, R₃ represents a hydrogen atom or an alkyl group having 1 to 3 carbon atoms, and R₄ represents a hydrogen atom or a methyl group or by Formula 2:

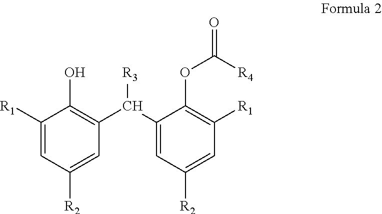

wherein each R₁ and R₂ independently represents an alkyl group having 1 to 5 carbon atoms, R₃ represents a hydrogen atom or an alkyl group having 1 to 3 carbon atoms, and R₄ group represents vinyl, vinyl ether or vinyl amine, 1-alkadiene, 1-alkene having 1 to 15 carbon atoms.

The drinking water pipe, preferably a pressure pipe, based on the polyolefin composition according to the invention has an improved protection against for example chlorine dioxide containing cold or hot water and consequently a longer life time. It is also possible to transport waste water or cooling water.

Preferably the amount of polyolefin in the composition is higher than 95.0 wt %.

Preferably the amount of bisphenol monoester in the composition is lower than 1.0 wt % and higher than 0.05 wt %.

More preferably the amount of bisphenol monoester in the composition is lower than of lower than 0.6 wt %.

Most preferably the amount of bisphenol monoester in the composition ranges between 0.05 and 0.4 wt %. This amount protects the pipe against chlorine dioxide during a long period.

The polyolefin may be selected from polyethylene such as a multimodal polyethylene for example a bimodal or trimodal polyethylene or polypropylene.

Preferably, the polyolefin is multimodal polyethylene.

More preferably, the polyolefin is bimodal polyethylene.

According to a preferred embodiment of the invention the pipe according to the invention produced with a polymer composition comprising polyethylene or propylene and a bisphenol monoester represented by Formula 1:

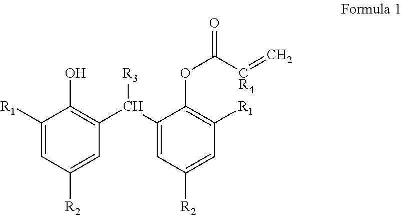

wherein each R₁ and R₂ independently represents an alkyl group having 1 to 5 carbon atoms, R₃ represents a hydrogen atom or an alkyl group having 1 to 3 carbon atoms, and R₄ represents a hydrogen atom or a methyl group.

In Formula 1 R₁ and R₂ independently represents an alkyl group having 1 to 5 carbon atoms. Suitable examples of the alkyl group include a methyl group, an ethyl group, an n-propyl group, an isopropyl group, an n-butyl group, an isobutyl group, a sec-butyl group, a tert-butyl group, an n-pentyl group, a tert-pentyl group and the like. In particular, R₁ is preferably an alkyl group having a tertiary carbon, that is, a tert-butyl group or a tert-pentyl group.

In Formula 1 R₃ represents a hydrogen atom or an alkyl group having 1 to 3 carbon atoms. Examples of the alkyl group include a methyl group, an ethyl group, an n-propyl group, and an isopropyl group, and among these, a methyl group is particularly preferable.

Suitable examples of the bisphenol monoester to be used in the present invention include 2-[1-(2-hydroxy-3,5-di-tert-pentylphenypethyl]-4,6-di-tert-pentylphenylacrylate, 2,4-di-tert-pentyl-6-[1-(3,5-di-tert-pentyl-2-hydroxyphenyl) ethyl]phenylacrylate, 2-tert-butyl-6-[1-(3-tert-butyl-2-hydroxy-5-methylphenyl)methyl]-4-methylphenylacrylate, 2-[1-(2-hydroxy-3,5-di-tert-pentylphenypethyl]-4,6-di-tert-pentylphenylmethacrylate, 2,4-di-tert-butyl-6-[1-(3,5-di-tert-butyl-2-hydroxyphenyl)ethyl]phenylacrylate, 2,4-di-tert-butyl-6-[1-(3,5-di-tert-butyl-2-hydroxyphenyl)ethyl]phenylmethacrylate, 2-tert-butyl-6-[1 -(3-tert-butyl-2-hydroxy-5-methylphenypethyl]-4-methylphenylacrylate, 2-tert-butyl-6-[1-(3-tert-butyl-2-hydroxy-5-methylphenyl)propyl]-4-methylphenylacrylate, 2-tert-butyl-6-[1 -(3-tert-butyl-2-hydroxy-5-propylphenyl)ethyl]-4-propylphenylacrylate and 2-tert-butyl-6-[1-(3-tert-butyl-2-hydroxy-5-isopropylphenyl)ethyl]-4-isopropylphenylacrylate.

According to a preferred embodiment of the invention the bisphenol monoester is selected from 2,4-di-tert-pentyl-6-[1-(3,5-di-tert-pentyl-2-hydroxyphenyl)ethyl]phenylacrylate or 2-[1-(2-hydroxy-3,5-di-tert-pentylphenyl)ethyl]-4,6-di-tert-pentylphenylacrylate and 2-tert-butyl-6-[1-(3-tert-butyl-2-hydroxy-5-methylphenyl)methyl]-4-methylphenylacrylate.

According to a further preferred embodiment of the invention the bisphenol monoester is 2-[1-(2-hydroxy-3,5-di-tert-pentylphenyl)ethyl]-4,6-di-tert-pentylphenylacrylate.

It is possible to apply compounds comprising more than one group according to Formula (1) and/or Formula (2).

According to a further preferred embodiment of the invention the pipe is produced with a composition comprising
(a) polyethylene or polypropylene
(b) bisphenol monoester
(c) polyphenolic compound and/or
(d) organic phosphite and/or phosphonate
   wherein the weight ratio (b): (c+d) ranges between 7:1 and 1:7.

Suitable polyphenolic compounds include for example tetrakis[methylene-3-(3′,5′-di-t-butyl-4-hydroxyphenyl)propionate] methane;1,1,3-tris(2-methyl-4-hydroxy-5-t-butylphenyl)butane; 1,3,5-trimethyl-2,4,6-tris(3,5-di-t-butyl-4-hydroxybenzyl)benzene, bis(3,3-bis(4′-hydroxy-3′-t-butylphenyl)butanoic acid]-glycol ester; tris(3,5-di-t-butyl-4-hydroxy benzyl)isocyanurate; 1,3,5-tris(4-t-butyl-2,6-dimethyl-3-hydroxy-benzyl)isocyanurate; 5-di-t-butyl-4-hydroxy-hydrocinnamic acid triester with 1,3,5-tris(2-hydroxyethyl)-s-triazine-2,4,6(1H, 3H, 5H)-trione; p-cresol/ dicyclopentadiene butylated reaction product; 2,6-bis(2′-bis-hydroxy-3′-t-butyl-5′-methyl-phenyl-4-methyl-phenol).

A preferred polyphenolic compound is 1,3,5-trimethyl-2,4,6-tris(3,5-di-t-butyl-4-hydroxybenzyl)benzene (Irganox 1330 supplied by BASF).

Suitable organic phosphites and phosphonites include for example triphenyl phosphite, diphenyl alkyl phosphites, phenyl dialkyl phosphites, tris(nonylphenyl) phosphite, trilauryl phosphite, trioctadecyl phosphite, distearyl pentaerythritol diphosphite, tris(2,4-di-tert-butylphenyl) phosphite, diisodecyl pentaerythritol diphosphite, bis(2,4-di-tert-butylphenyl) pentaerythritol diphosphate, bis(2,6-di-tert-butyl-4-methylphenyl) pentaerythritol diphosphate, bisisodecyloxy-pentaerythritol diphosphite, bis(2,4-di-tert-butyl- 6-methylphenyl) pentaerythritol diphosphite, bis(2,4,6-tri-tert-butylphenyl) pentaerythritol diphosphite, tristearyl sorbitol triphosphite, tetrakis(2,4-di-tert-butylphenyl) 4,4′- biphenylenediphosphonite, 6-isooctyloxy-2,4,8,10-tetra-tert-butyl-12H-dibenzo[d,g]-1,3,2-dioxaphosphocin, 6-fluoro-2,4,8,10-tetra-tert-butyl-12-methyldibenzo[d,g]-1,3,2-dioxaphosphocin, bis(2,4-di-tert-butyl-6-methylphenyl) methyl phosphite, bis(2,4-di-tert-butyl- 6-methylphenyl) ethyl phosphite.

A preferred phosphite is tris(2,4-di-tert-butylphenyl) phosphite (Irgafos 168 supplied by BASF).

According to another preferred embodiment of the invention the pipe is produced with a composition comprising
(a) multimodal polyethylene
(b) bisphenol monoester
(c) polyphenolic compound and/or
(d) organic phosphite and/or phosphonate
   wherein the weight ratio (b): (c+d) ranges between 7:1 and 1:7.

Preferably (b, (c) and (d) are added during the granulation step of the multimodal, for example bimodal, high density polyethylene powder.

According to a preferred embodiment of the invention the components are added to the polyethylene resin while the polyethylene is in a molten state during extrusion.

The components may be added together and may be added separately.

Preferably the components are added in one step.

The multimodal ethylene polymer may be an ethylene homo- or copolymer.

The multimodal ethylene grades to be applied in pipe applications may comprise additives such as for example carbon black, pigments, stearates, a UV stabilizer for example a sterically hindered amine, fillers, minerals,lubricants and/or other stabilisers.

The production processes for bimodal high density polyethylene (HDPE) are summarised at pages 16-20 of “PE 100 Pipe systems” (edited by Bromstrup; second edition, ISBN 3-8027-2728-2).

The production of bimodal high density polyethylene (HDPE) via a low pressure slurry process is described by Alt et al. in “Bimodal polyethylene-Interplay of catalyst and process” (Macromol.Symp. 2001, 163, 135-143). Bimodal high density polyethylene may be produced via a low pressure slurry process for the production of comprising a polymerisation stage, a powder drying stage, an extrusion and pellet handling stage, a recycling stage and a wax removal unit. In a two stage cascade process the reactors may be fed continuously with a mixture of monomers, hydrogen, catalyst/co-catalyst and diluent recycled from the process. In the reactors, polymerisation of ethylene occurs as an exothermic reaction at pressures in the range between for example 0.5 MPa (5 bar) and 1MPa (10 bar) and at temperatures in the range between for example 75° C. and 88° C. The heat from the polymerisation reaction is removed by means of cooling water. The characteristics of the polyethylene are determined amongst others by the catalyst system and by the concentrations of catalyst, co monomer and hydrogen. The production of bimodal high density polyethylene (HDPE) via a low pressure slurry process may also be performed via a three stage process.

The concept of the two stage cascade process is elucidated at pages 137-138 by Alt et al. “Bimodal polyethylene-Interplay of catalyst and process” (Macromol. Symp. 2001, 163). The reactors are set up in cascade with different conditions in each reactor including low hydrogen content in the second reactor. This allows for the production of HDPE with a bimodal molecular mass distribution and defined co monomer content in the polyethylene chains.

Suitable catalysts for the production of multimodal polyethylene include Ziegler Natta catalysts, chromium based catalysts and single site metallocene catalysts. In all potential possible technologies the process and the catalyst have to form a well-balanced system. The catalyst is crucial for the polymerisation reaction of multimodal polyethylene. By cooperation of process and catalyst a definite polymer structure is produced.

US20130035426 discloses a composition comprising a specific compound represented by a very broad formula, trehalose and thermoplastic polymer selected from 87 polymers to improve process stability. US20130035426 is not related to a pipe with improved service lifetime for the transportation of water containing chlorinated disinfectants.

DE19629429 is directed to a process to produce grafted polyolefins in the presence of silane compounds which may comprise bisphenol monoesters. DE19629429 provides a solution for problems with respect to a graft process in the presence of siloxane bridges. DE19629429 is not related to a pipe with improved service lifetime for the transportation of water containing chlorinated disinfectants.

The invention will be elucidated by means of the following non-limiting examples.

### EXAMPLES

SABIC Vestolen A5924 (Resin A) used as base polymer in all examples was a bimodal high density polyethylene with MFR₅ of 0.24 g/10min and density 958 kg/m³.

### Examples I-II and Comparative Examples A-C

The Examples and Comparative Examples A-C use different additive packages in combination with Resin A to protect the polyethylene from attack by chlorine dioxide (see Table 1). The components as indicated in Table 1 were mixed at 245 degrees Celcius using a twin screw extruder.

| | | Calcium | Carbon | Irganox | Irgafos | Bisphenol | Irganox | |
|---|---|---|---|---|---|---|---|---|
| | Resin A | stearate | black | 1010 | 168 | monoester | 1330 | DHT4A |
| Composition | wt % | ppm | wt % | ppm | ppm | ppm | ppm | ppm |
| A | 97 | 2000 | 2.5 | 2000 | 1000 | 0 | 0 | 0 |
| B | 96.15 | 2000 | 2.5 | 2000 | 2500 | 0 | 5000 | 2000 |
| C | 96.44 | 2000 | 2.5 | 2000 | 2000 | 0 | 3200 | 1400 |
| I | 96 | 2000 | 2.5 | 2000 | 2500 | 1500 | 5000 | 2000 |
| II | 96.34 | 2000 | 2.5 | 2000 | 2000 | 1000 | 3200 | 1400 |

wherein:
Irganox 1010: Tetrakis [methylen-3-(3′,5′)-di-t-butyl-4′-hydroxyphenyl) propionate] methane commercially available from Ciba Speciality Chemicals,
Bisphenol monoester: 2-[1-(2-Hydroxy-3,5-di-tert-pentylphenyl)ethyl]-4,6-di-tert-pentylphenyl acrylate;
Irganox 1330: 1,3,5-Trimethyl-2,4,6-tris(3,5-di-tert-butyl-4-hydroxybenzyl)benzene;
Irgafos 168: Tris(2,4-di-tert-butylphenyl) phosphite;
DHT-4A ®, commercially available hydrotalcite from Kisuma Chemicals.
Resin A: SABIC Vestolen A5924; bimodal high density polyethylene with MFR₅ of 0.24 g/10 min and density 958 kg/m³.

Compounds were compression molded using ISO1872-2 resulting in plaques, which were cut to ISO527-1A tensile bars (4 mm thick).

Ageing test

The tensile bars were aged in a continuous water flow at a temperature of 40° C. with a chlorine dioxide concentration maintained at 1 mg/L and a pH maintained at 7.2. Flow rate was regulated at 200 L/h. Water hardness was regulated to 20° F. A constant fresh water flow was added during testing allowing full renewal of the testing water each 4 hrs.

The Compression Molded Samples were Aged for 1000 hrs.

Tensile tests according to Plastics—Determination of tensile properties ISO527-1 at room temperature at a strain rate of 50 mm/min on aged and non-aged tensile bars were performed to determine the residual elongation at break for the aged samples and reported in Table 2.

TABLE 2

| | Elongation @ break | Elongation @ break |
|---|---|---|
| Composition | before ageing in % | after ageing in % |
| A | 466 | 33 |
| B | 301 | 259 |
| C | 463 | 32 |
| I | 463 | 462 |
| II | 346 | 136 |

From Table 2 it can be concluded that Examples I and II demonstrate significantly higher elongation at break after being exposed to water containing chlorine dioxide than Comparative Example A.

### Comparing

Comparative Example B to Example I and

Comparative Example C to Example II shows that the effect of adding bisphenol monoester had an additional profound effect on the elongation at break as obtained after exposure to water containing chlorine dioxide.

1. A pipe for the transport of water with resistance to chlorinated disinfectants, wherein the pipe is produced with a polymer composition comprising polyethylene or propylene and a bisphenol monoester that is
represented by Formula 1: 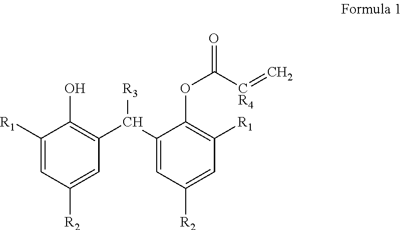
wherein each R₁ and R₂ independently represents an alkyl group having 1 to 5 carbon atoms, R₃ represents a hydrogen atom or an alkyl group having 1 to 3 carbon atoms, and R₄ represents a hydrogen atom or a methyl group; or
represented by Formula 2: 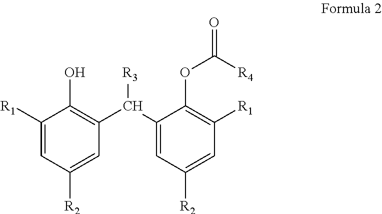
wherein each R₁ and R₂ independently represents an alkyl group having 1 to 5 carbon atoms, R₃ represents a hydrogen atom or an alkyl group having 1 to 3 carbon atoms, and R₄ represents vinyl, vinyl ether, vinyl amine, 1-alkadiene, or 1-alkene having 1 to 15 carbon atoms.

2. The pipe according to claim 1 characterised in that the bisphenol monoester is a bisphenol monoester according to Formula 1.

3. The pipe according to claim 1 characterised in that the composition comprises a polyphenolic compound and/or an organic phosphite and/or a phosphonite.

4. The pipe according to claim 1 characterised in that the amount of bisphenol monoester in the composition is lower than 1.0 wt %.

5. The pipe according to claim 4 characterised in that the amount of bisphenol monoester in the composition is lower than 0.6 wt %.

6. The pipe according to claim 5 characterised in that the amount of bisphenol monoester in the composition ranges between 0.05 and 0.4 wt %.

7. The pipe according to claim 1 any one of claim 1 characterised in that the polyethylene is a multimodal polyethylene.

8. The pipe according to claim 7 characterised in that multimodal polyethylene is bimodal polyethylene.

9. The pipe according to claim 1 characterised in that the bisphenol monoester is selected from 2,4-di-tert-pentyl-6-[1-(3, 5 -di-tert-pentyl-2-hydroxyphenyl)ethyl]phenylacrylate, 2-[1-(2-hydroxy-3,5 -di-tert-pentylphenyl)ethyl]-4,6-di-tert-pentylphenylacrylate, and 2-tert-butyl-6-[1-(3-tert-butyl-2-hydroxy-5-methylphenyl)methyl]-4-methylphenylacrylate.

10. The pipe according to claim 9 characterised in that the bisphenol monoester is 2-[1-(2-hydroxy-3,5-di-tert-pentylphenyl)ethyl]-4,6-di-tert-pentylphenylacrylate.

11. The pipe according to claim 3 characterised in that the polyphenolic compound is 1,3,5-trimethyl-2,4,6-tris(3,5-di-t-butyl-4-hydroxybenzyl)benzene.

12. The pipe according to claim 3 characterised in that the phosphite is tris(2,4-di-tert-butylphenyl) phosphite.

13. The pipe according to claim 1, wherein the pipe is produced with a composition comprising
(a) the polyethylene or polypropylene,
(b) the bisphenol monoester
(c) a polyphenolic compound, and/or
(d) an organic phosphite and/or an organic phosphonate.
wherein the weight ratio (b): (c+d) ranges between 7:1 and 1:7.

14. The pipe according to claim 13, wherein the pipe is produced with a composition comprising
(a) a bimodal polyethylene,
(b) the bisphenol monoester,
(c) the polyphenolic compound, and/or
(d) the organic phosphite and/or the organic phosphonate,
wherein the weight ratio (b): (c+d) ranges between 7:1 and 1:7.

15. The pipe according to claim 14 characterised in that
the bisphenol monoester is 2-[1-(2-hydroxy-3,5-di-tert-pentylphenyl)ethyl]-4,6-di-tert-pentylphenylacrylate, and
the polyphenolic compound is 1,3,5-trimethyl-2,4,6-tris(3,5-di-t-butyl-4-hydroxybenzyl)benzene,
the phosphite is tris(2,4-di-tert-butylphenyl) phosphite.
